(19) United States
(12) Patent Application Publication
(10) Pub. No.: US 2012/0130061 A1
(43) Pub. Date: May 24, 2012

(54) Method F Method For Isolating And Purifying Nucleic Acids

(75) Inventors: Ralf Himmelreich, Langenfeld (DE); Sabine Werner, Duesseldorf (DE)

(73) Assignee: QIAGEN GMBH, Hilden (DE)

(21) Appl. No.: 13/061,916

(22) PCT Filed: Sep. 2, 2009
(86) PCT No.: PCT/EP09/61360

§ 371 Date: Mar. 29, 2011

(30) Foreign Application Priority Data
Sep. 3, 2008 (EP) 08163623.5

(51) Int. Cl. C07H 1/06; C07H 21/04; C09K 3/00; C07H 1/08; C07C 43/13; C07H 21/00; C07H 21/02

(52) U.S. Cl. 536/25.41; 568/622; 252/182.12

(57) ABSTRACT

The present invention relates to a method for the isolation and purification of nucleic acids by elution of nucleic acids from nucleic acid-containing samples and biological materials. The present invention further relates to a kit for carrying out the method of the invention.

The present invention relates to a method for the isolation and purification of nucleic acids by elution of the nucleic acids from nucleic acid-containing samples, and biological materials. Furthermore, the present invention relates to a kit for carrying out the method of the present invention.

An efficient method for the isolation and purification of nucleic acids, already known in the prior art, is based on the adsorption of nucleic acids on glass or silica particles in the presence of chaotropic salts, and the subsequent recovery of the adsorbed nucleic acids (Vogelstein, B. and Gillespie, D. (1979); “Preparative and analytical purification of DNA from agarose”, Proc. Natl. Acad. Sci. USA 76: 615-619). According to this method, DNA is isolated and purified on agarose using high concentrations of chaotropic salts, such as sodium iodide, sodium perchlorate or guanidinium thiocyanate. The RNA or DNA may also be isolated or purified from various mixtures (Boom, R. (1990); “Rapid and simple method for purification of nucleic acids”, J. Clin. Microbiol. 28: 495-503).

After purification, nucleic acids are often used in polymerase chain reaction (PCR). The PCR amplifies polynucleic acids in a sequence-specific manner and is therefore widely used in genetic diagnosis or DNA diagnosis. The use of PCR technology in clinical routine methods entails several problems. It is known that inhibitory substances that have not been removed from the purified nucleic acid preparation may inhibit the PCR. Such inhibitory substances are, e.g., hemoglobin and surfactants, which were used in the nucleic acid extraction process. Against this background it is apparent that the methods for the extraction and purification of nucleic acids are extremely important and relevant (Oshima et al., JJCL A, 22(2) 145-150 (1997)).

Methods for the extraction and purification of nucleic acids are frequently automated. The prior art already knows automated nucleic acid extraction methods, as described, e.g., in JP-A-107854/1999 and in JP-A-266864/1999. In most methods for the isolation and purification of nucleic acids, a solution containing a high concentration of salts and a high concentration of alcohol, and in which the nucleic acids are present, is brought into contact with an adsorption surface. Here, the adsorption surface may be a column. Subsequently the nucleic acids are adsorbed on this surface and later eluted using solutions containing less concentrated salt solutions.

The problem with most methods for the isolation and purification of nucleic acids consists in that the yield of nucleic acids is comparatively small. A further problem is that, according to the IATA (International Air Transportation Association) Regulations, ethanol-containing solutions are classified as dangerous materials (HAZMAT; hazardous materials). According to the IATA Regulations, all products, materials and goods are categorized in nine main classes. Where goods are classified as dangerous, additional fees and taxes become due for air transport. It was therefore the object of the present invention to replace as far as possible ethanol (or isopropanol) in the method for the purification and extraction of nucleic acids to facilitate the isolation and purification of nucleic acids, to provide an ethanol-free method, and to facilitate the transport of air cargo.

The prior art discloses substitutes for alcohol in methods for the purification of nucleic acids, which, however, solve the above discussed problems only in part (US 2004/0167324). The majority of the substances described therein either fall under the HAZMAT IATA Regulations or have an acrid smell so that they may only be used in a fume hood.

To better solve the above mentioned problems there was a need for further alcohol substitutes in methods for the isolation and purification of nucleic acids.

The present invention relates to a method for the extraction of nucleic acids from a solution, comprising the steps of:
(a) adding a binding mediator to the nucleic-acid containing solution,
(b) contacting the solution containing the binding mediator and the nucleic acids with a surface under chaotropic and/or high-salt conditions,
(c) binding or adsorption of the nucleic acids to a surface,
(d) washing the surface with a wash buffer,
(e) recovery of the nucleic acids adsorbed on the surface by elution,
   characterized in that the binding mediator is selected from the group comprising diethylene glycol monoethyl ether, diethylene glycol monoethyl ether acetate, furfuryl alcohol, poly(1-vinylpyrrolidon-co-2-dimethyl-aminoethyl-methacrylate), poly(2-ethyl-2-oxazoline), poly(4-ammonium-styrene-sulfonic acid), tetraethylene glycol dimethyl ether, tetraethylene glycol, tetrahydrofurfuryl-polyethylene glycol 200 and triethylene glycol monoethyl ether.

In the binding process of the nucleic acid preparation, the person skilled in the art can also successfully replace ethanol by mixtures of the mentioned binding mediators. Since ethanol-containing solutions of up to 24% (vol/vol) are not classified as HAZMAT, it is also possible to use mixtures of the binding mediators with ethanol.

If not otherwise stated, the concentrations mentioned in the invention are volume percentages (percent by volume, % by volume, % (v/v)). Concentrations in weight percent are represented by percent by weight (% by weight, % (w/v)).

Preferably, the binding mediators are present in the following concentrations:
diethylene glycol monoethyl ether (DGME) [CAS 111-90-0]—concentration range 70-99%, preferred concentration 99.0%; in combination with ethanol: 60-80% DGME and 16-24% ethanol
diethylene glycol monoethyl ether acetate (DGMEA) [CAS 112-15-2]—concentration range 70-99%, preferred concentration 99.0%, in combination with ethanol: 60-80% DGMEA and 16-24% ethanol
furfuryl alcohol [CAS 98-00-0]—concentration range 20-30%, preferred concentration 30%
poly(1-vinylpyrrolidone-co-2-dimethyl-aminoethyl-methacrylate) [CAS 30581-59-0)—concentration range 3-5%, preferred concentration 5%
poly(2-ethyl-2-oxazoline) [CAS 25805-17-8]—concentration range 9-15% (w/v), preferred concentration 12%; in combination with ethanol: 22.5% (w/v) and 16-24% (v/v) ethanol
poly(4-ammoniumstyrene sulfonic acid)—concentration range 8-22% (w/v), preferred concentration 12%; in combination with ethanol: 8-22 (w/v) and 24% (v/v) ethanol
tetraethylene glycol dimethylether [CAS 143-24-8]—concentration range 70-98%, preferred concentration 98%; in combination with ethanol: 73.5% and 24% ethanol
tetraglycol[CAS 9004-76-6]—preferred concentration with ethanol: 75% and 16-24% ethanol
tetrahydrofurfuryl polyethylene glycol 200 [CAS 31692-85-0]—concentration range 70-100%, preferred concentration 100%
triethylene glycol monoethyl ether [CAS 112-50-5]—concentration range 70-90%, preferred concentration 90%

Most of the binding mediators of the present invention are classified by IATA as not dangerous. In addition, good yields have been achieved with the binding mediators of the invention (see Example 9).

The nucleic acid-containing solution can be obtained by lysis from a biological sample material containing nucleic aid. This sample material may be, e.g., blood, tissue, smear preparations, bacteria, cell suspensions, urine and adherent cells. The nucleic acid-containing material may be human, animal or plant material.

The nucleic acid-containing solution may be obtained from a biochemical nucleic acid modification reaction or from polymerase chain reactions.

For example, the nucleic acid can be genomic DNA, total DNA, or short double-stranded DNA fragments.

In a preferred embodiment, the nucleic acid is genomic DNA.

In another preferred embodiment, the nucleic acid is total RNA.

In a further preferred embodiment, the nucleic acids are short double-stranded DNA fragments.

In a preferred embodiment, the nucleic acid-containing solution has been obtained by lysis from a nucleic acid-containing material.

In another preferred embodiment, the nucleic-acid containing solution has been obtained from a biochemical nucleic acid modification reaction.

Chaotropic conditions are achieved by adding chaotropic substances. Chaotropic substances are chemical substances which disrupt ordered hydrogen bonding in aqueous solutions. They thus reduce the hydrophobic effect and have a denaturing effect on proteins, since the driving force behind protein folding is the clustering of hydrophobic amino acids in water. Examples of chaotropic substances are barium salts, guanidinium hydrochloride, thiocyanates, such as guanidinium thiocyanate, perchlorates, or even sodium chloride. Depending on their solubility product, chaotropic salts may be used in concentration ranges between 1 M and 8 M.

High-salt conditions means highly concentrated salt solutions, wherein the salt concentration in the solution is at least 1 M, and preferably 1-4 M.

However, it is also possible to take alternative measures to reach chaotropic or high-salt conditions achieving the same effect, i.e. the binding of the nucleic acids to be purified to the surface.

The surface on which the nucleic acids are adsorbed is based on materials selected from the following group: silica materials, carboxylated surfaces, zeolites and titanium dioxide.

According to the present invention, the method of the invention is preferably characterized in that chaotropic conditions are achieved by the addition of chaotropic salts, such as potassium iodide, guanidinium hydrochloride, guanidinium thiocyanate or sodium chloride, to the nucleic-acid containing solution.

Preferably, surfactants are added to the nucleic acid-containing solution. These surfactants are preferably used in concentration ranges from 0.1% by volume to 10% by volume. In addition, agents preventing foam formation (antifoams) may be added, preferably in a range from 0.01 to 1% by weight.

Wash buffers and elution buffers that can be employed in the methods of the invention are known to the skilled person.

Wash buffers contain organic solvents, such as alcohol. Wash buffers remove the other components from the nucleic acid-containing solutions (other than the nucleic acids).

Elution buffers are usually buffered low-salt solutions with a neutral to slightly alkaline pH value (e.g., buffer TE of the company QIAGEN GmbH, Hilden). The skilled person sometimes also uses distilled water.

The present invention relates to a reagent kit for the extraction of nucleic acids from a solution, comprising
a solution 1 comprising the binding mediator selected from the group comprising diethylene glycol monoethyl ether, diethylene glycol monoethyl ether acetate, furfuryl alcohol, poly(1-vinylpyrrolidone-co-2-dimethyl-aminoethyl-methacrylate), poly(2-ethyl-2-oxazoline), poly(4-ammonium-styrene-sulfonic acid), tetraethylene glycol dimethyl ether, tetraethylene glycol, tetrahydrofurfuryl polyethylene glycol 200 and triethylene glycol monoethyl ether, and optionally
a solution 2 comprising (a) wash buffer(s), and optionally
a solution 3 comprising an eluant.

In addition to the mentioned binding mediators, a kit of the company QIAGEN for the purification of nucleic acids from biochemical nucleic acid modification reactions would, for example, further contain the following components:
Adsorptive media: QIAGEN (QIAamp®; RNeasy®, QIAquick®) Spin Columns or magnetic silica particles (“MagAttract® Suspension G”)
Binding buffer: consisting of a chaotropic salt and binding mediators
Wash buffer: “Buffer PE” (see Table I for the description of the buffer)
Elution buffer: “Buffer AE”, “Buffer EB”; “Buffer TE”; “RNase-free water”

In addition to the just mentioned binding mediators, a kit of the company QIAGEN for the purification of nucleic acids from biological sample materials would comprise, e.g., the following components:
Adsorptive media: QIAGEN (QIAamp®; RNeasy®, DNeasy®; QIAprep®) Spin Columns or magnetic silica particles (“MagAttract® Suspension G”)
Lysis buffer: “Buffer AL”; “Buffer RLT”; Buffer ATL”, Buffer ML”; Buffer AP1”; or other buffers which are already commercially available
Protease: “QIAGEN Protease”; proteinase K; lysozyme and other proteolytic enzymes
Wash buffer: “Buffer AW1”; “Buffer AW2”; Buffer RW1”; Buffer RPE”; or other buffers which are already commercially available
Elution buffer: “Buffer AE”, “Buffer EB”; “Buffer TE”; “RNase-free water”

Corresponding lysis buffers are known to the skilled person. They usually contain detergents, chelators for divalent cations, pH buffer substances and chaotropic salts.

In a preferred embodiment, the reagent kit according to the present invention for the extraction of nucleic acids may comprise wash buffers and elution buffers, as described in WO 99/22021, EP 1 121 460 and U.S. Pat. No. 7,074,916. The wash buffers and elution buffers described therein are part of the present disclosure.

In a preferred embodiment, the reagent kit according to the present invention for the extraction of nucleic acids may comprise as eluant, e.g., “buffer TE” or even distilled water.

In a preferred embodiment, the reagent kit according to the present invention for the extraction of nucleic acids from a solution contains a chaotropic salt in a buffer solution. The kit thus contains, for example, a chaotropic buffer, a lysis buffer and a binding mediator.

Preferably, the chaotropic salt is selected from the group comprising sodium iodide, guanidinium hydrochloride, guanidinium thiocyanate; sodium perchlorate and sodium chloride.

The present invention further relates to the use of the reagent kits according to the present invention for the purification of nucleic acids from biological materials, such as blood, tissue, smear preparations, bacteria, cells suspensions and adherent cells.

The present invention also relates to the use of reagent kits according to the present invention for the purification of nucleic acids from biochemical reactions, PCR reactions and in vitro nucleic acid modification reactions.

Unless otherwise stated, the products, buffers and protocols (process instructions) described in the present application are published documents and commercially available products of the company QIAGEN GmbH, Hilden, Germany.

### DESCRIPTION OF THE FIGURES

FIG. 1: Behavior of poly(2-ethyl-2-oxazoline) and TetraGlyme in the QIAamp® 96 Spin Blood Protocol.

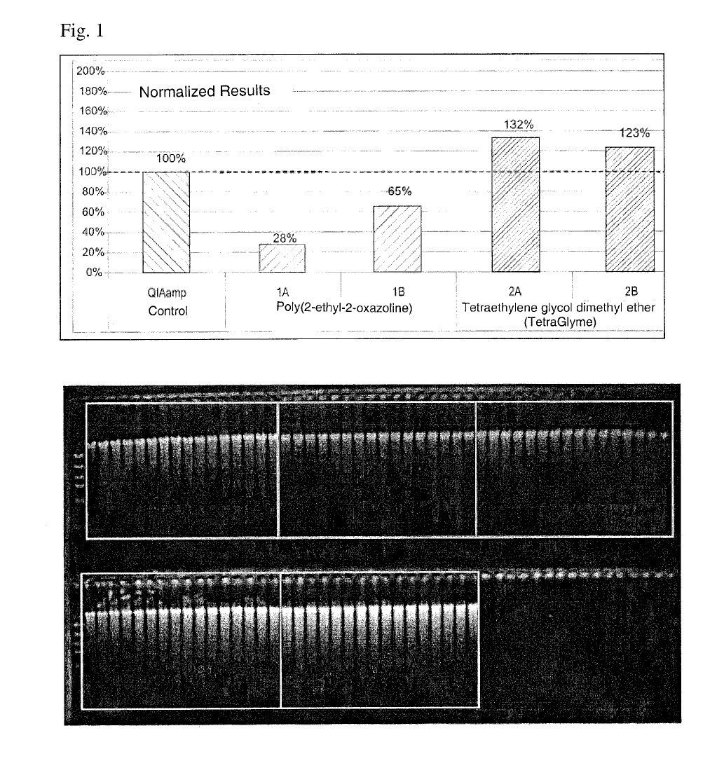

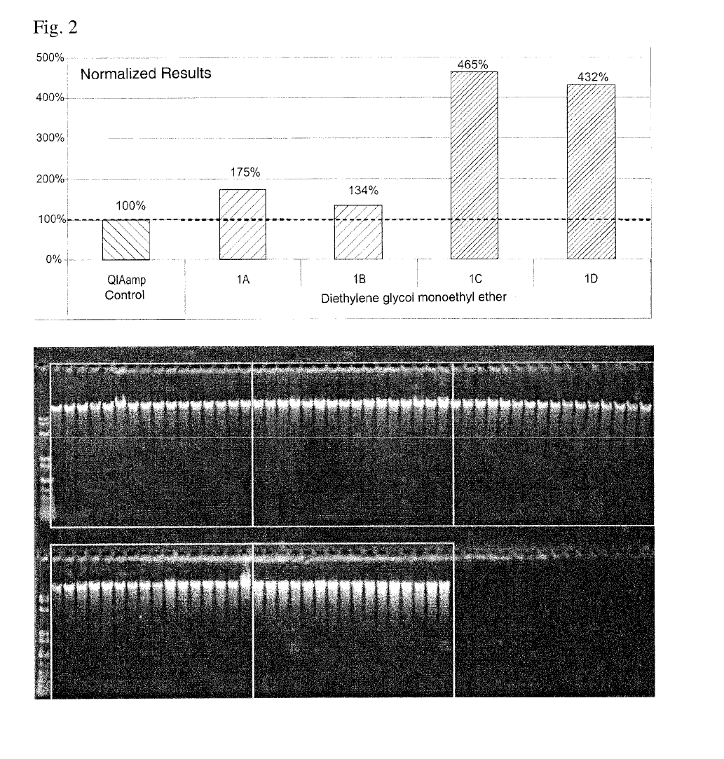

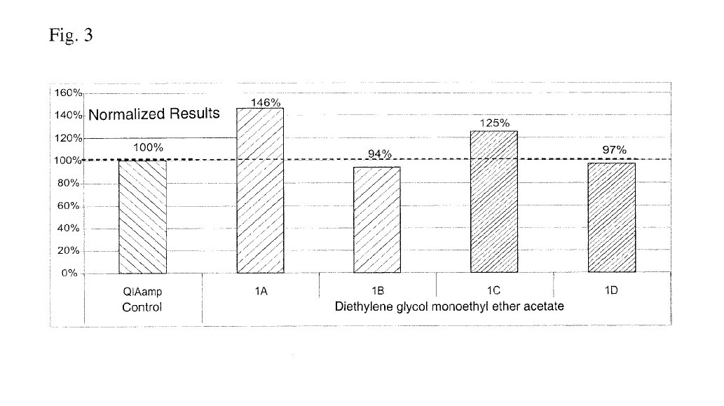

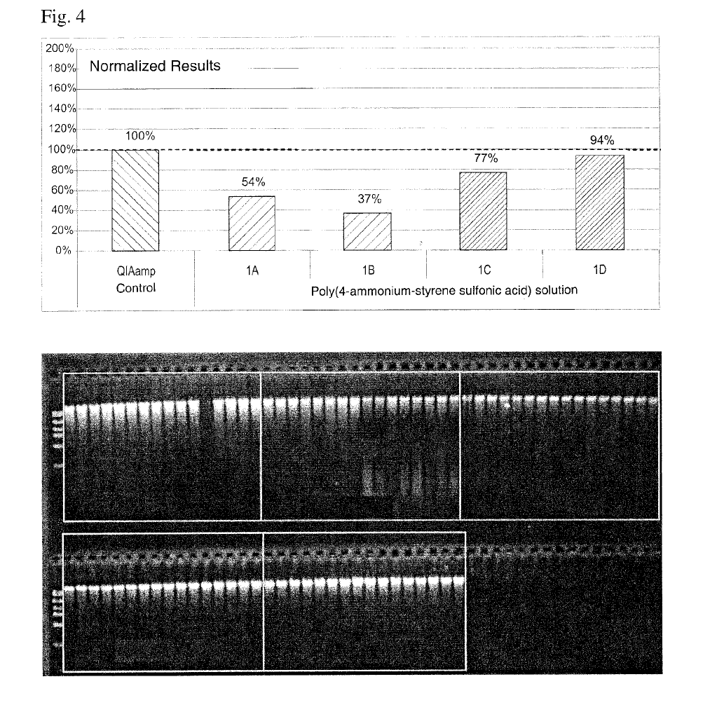

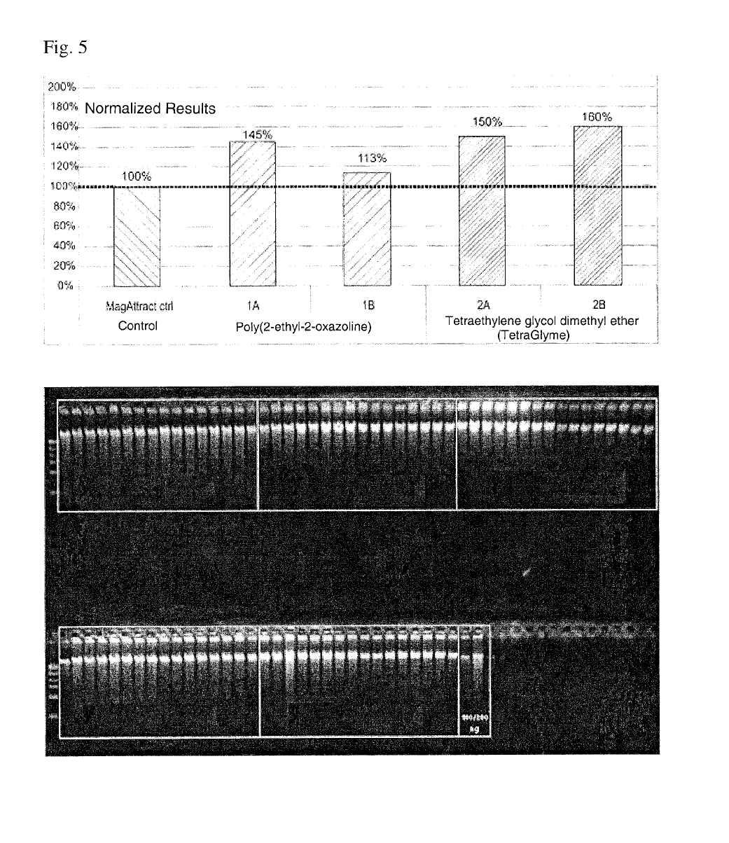

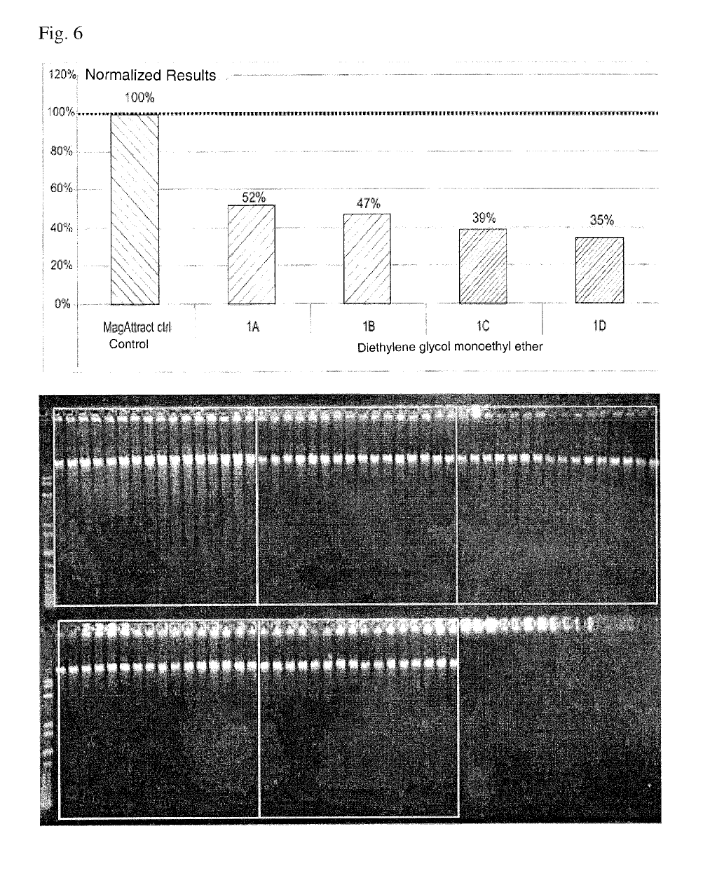

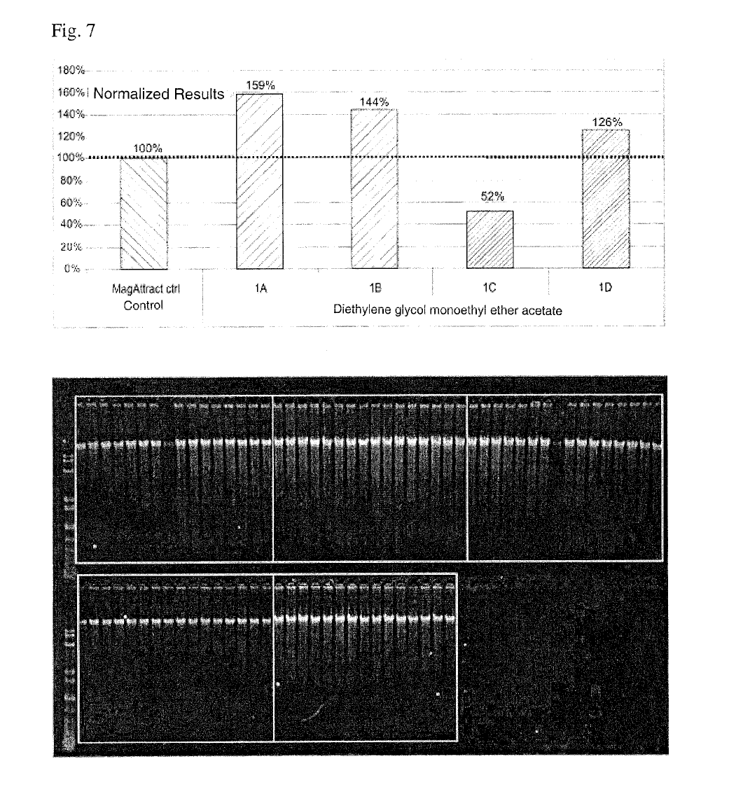

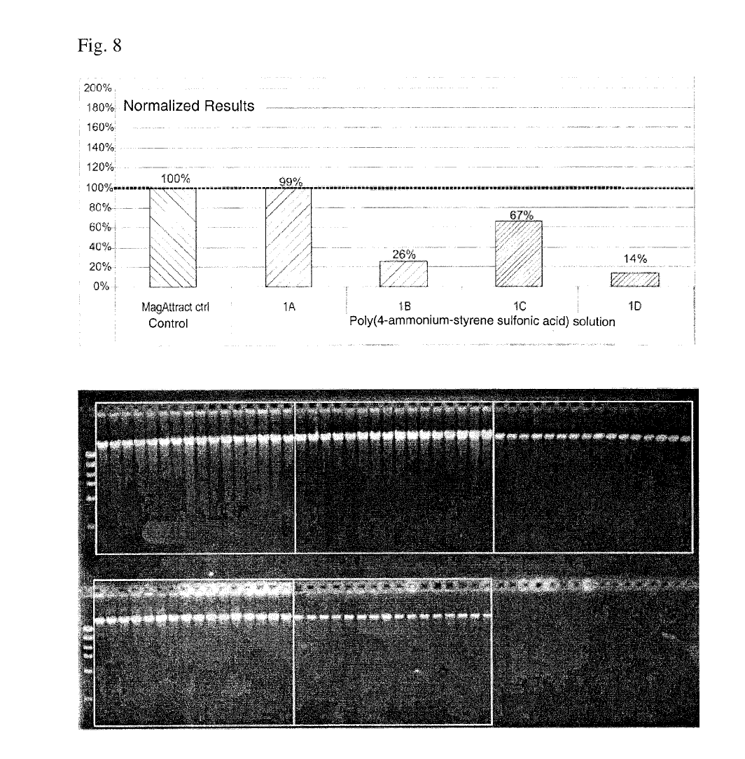

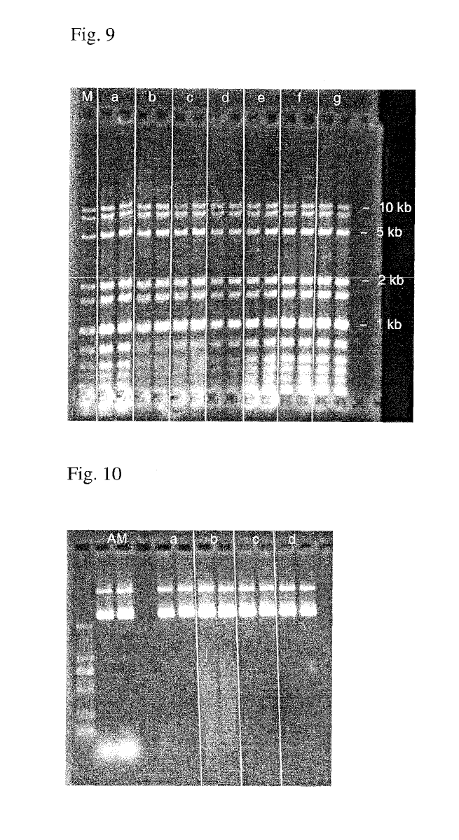

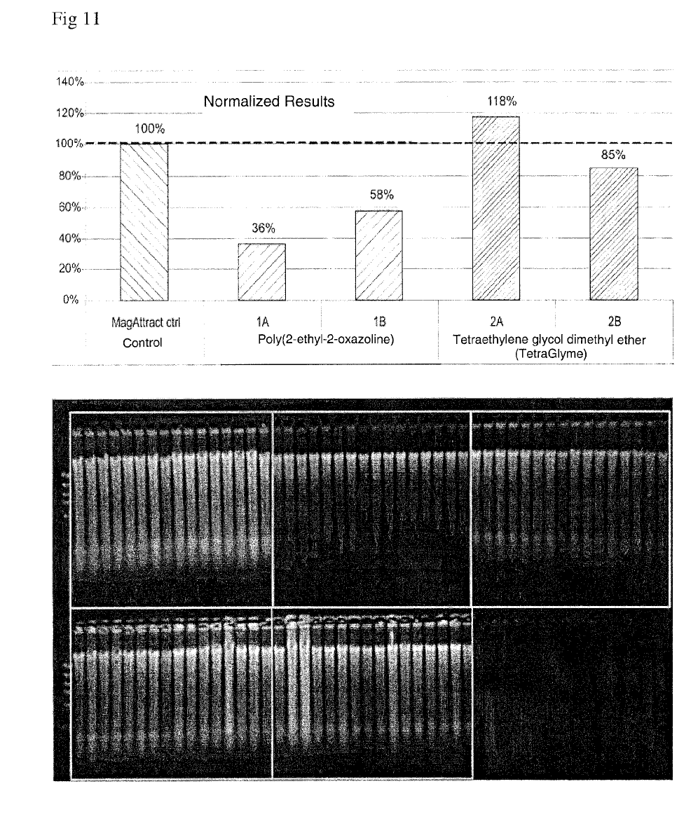

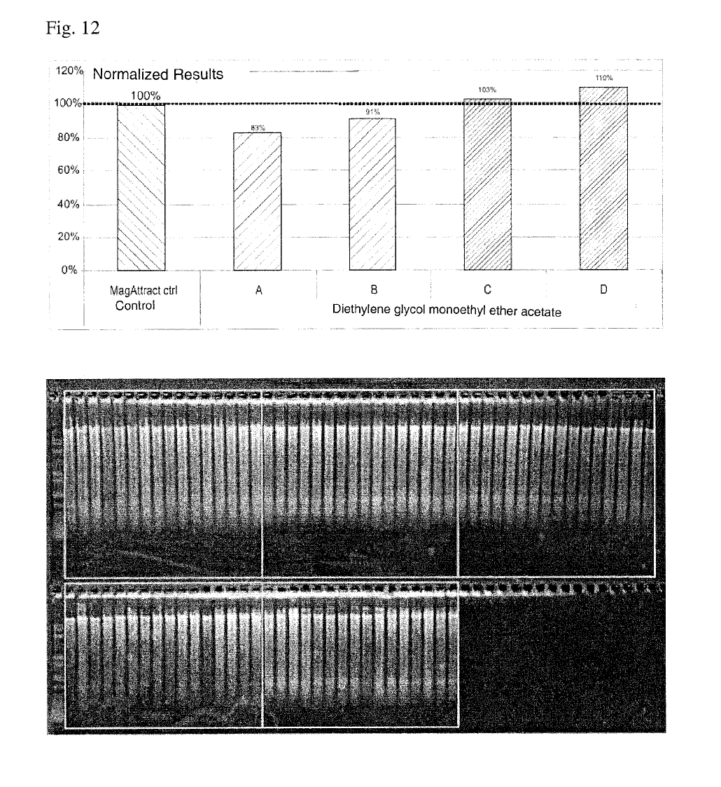

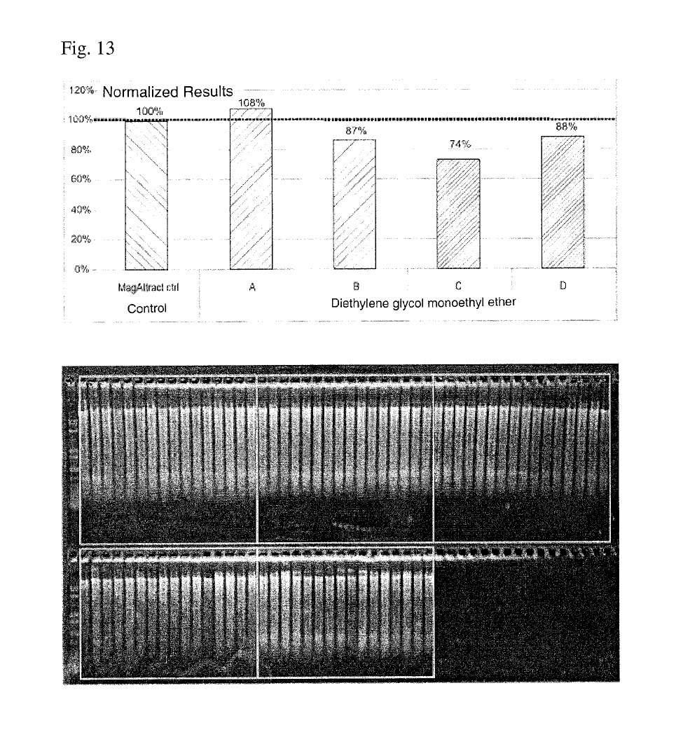

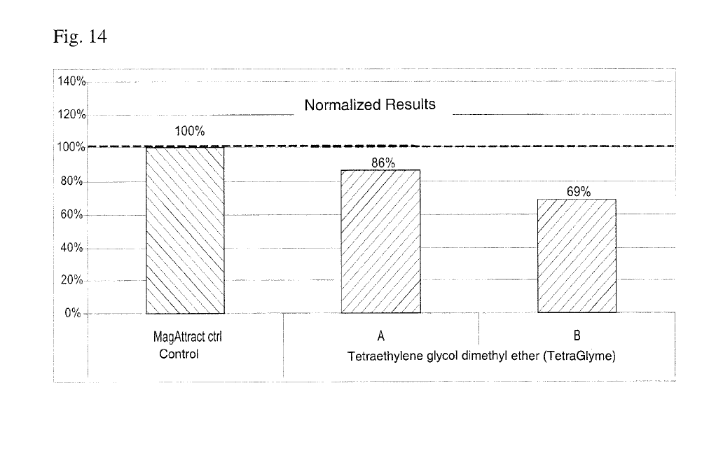

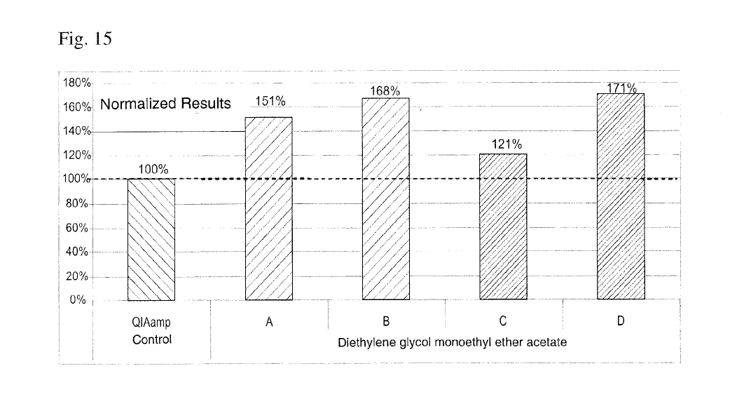

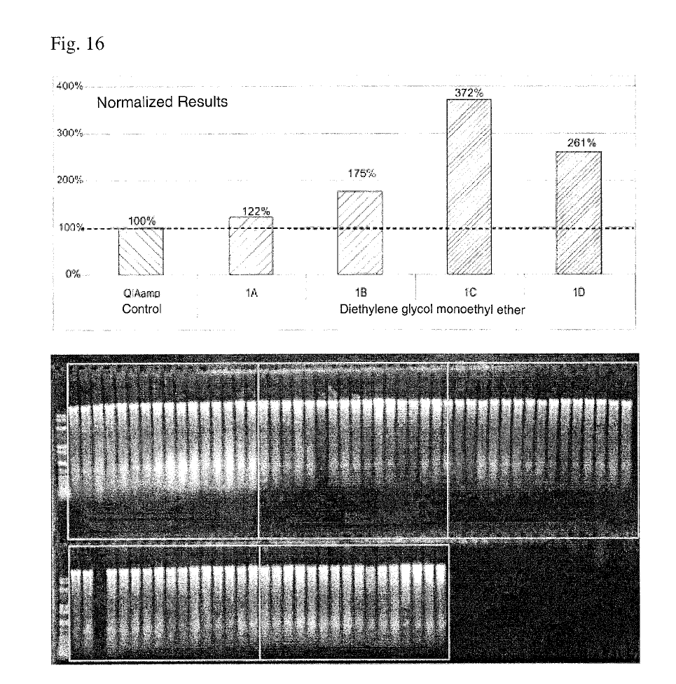

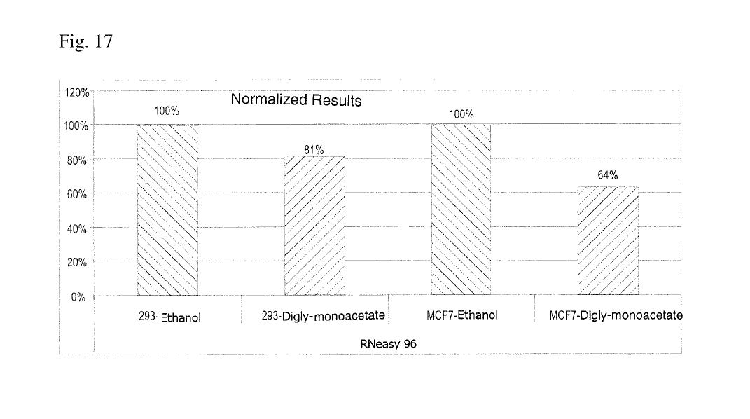

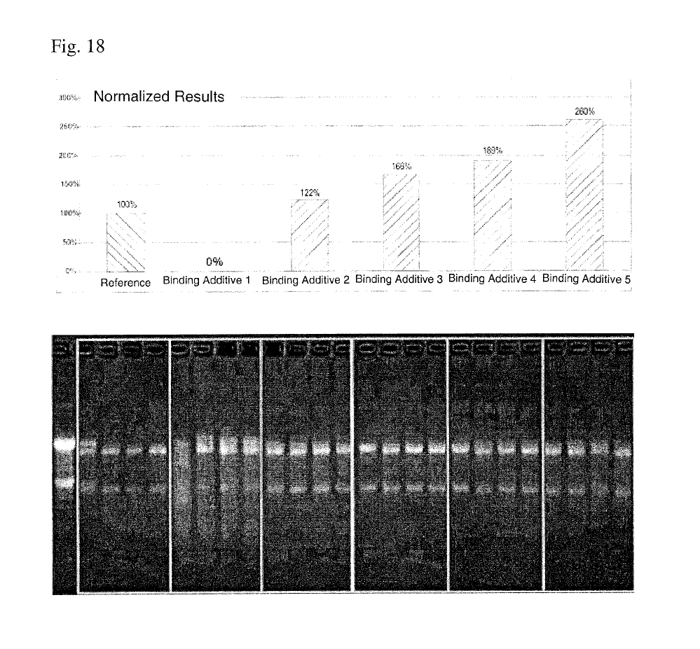

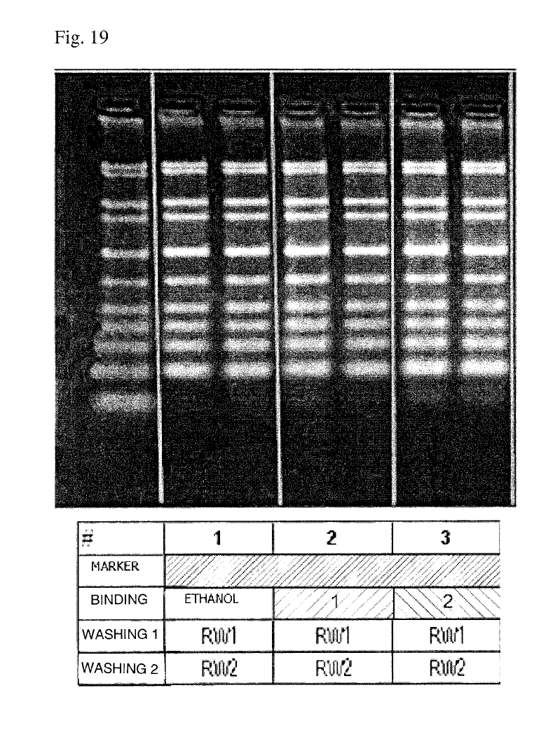

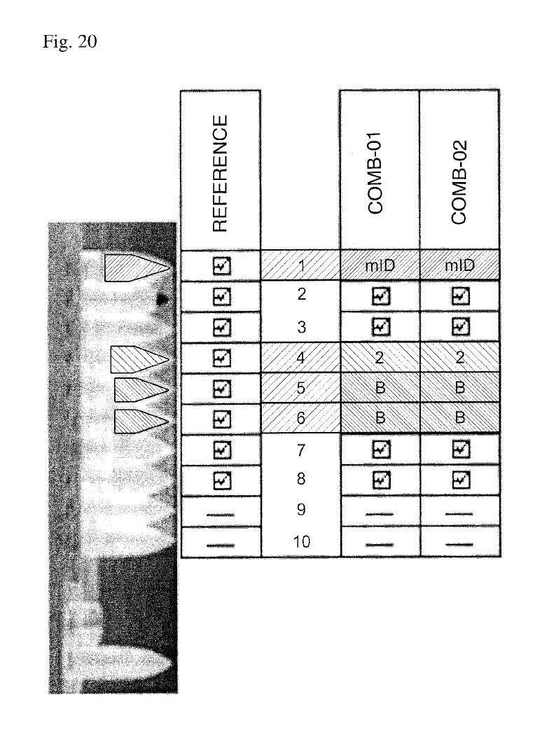

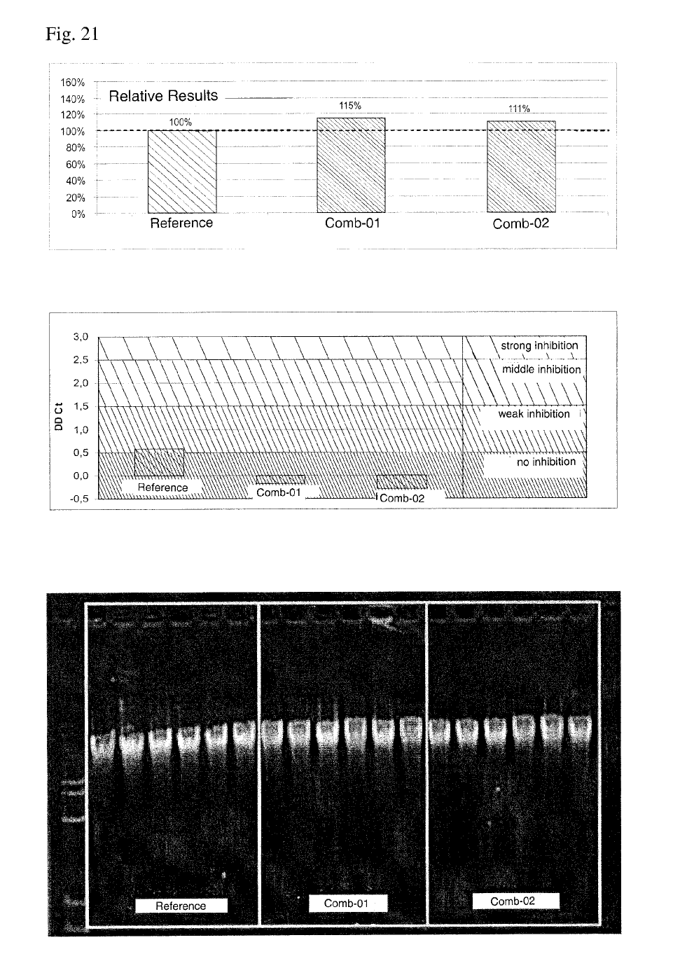

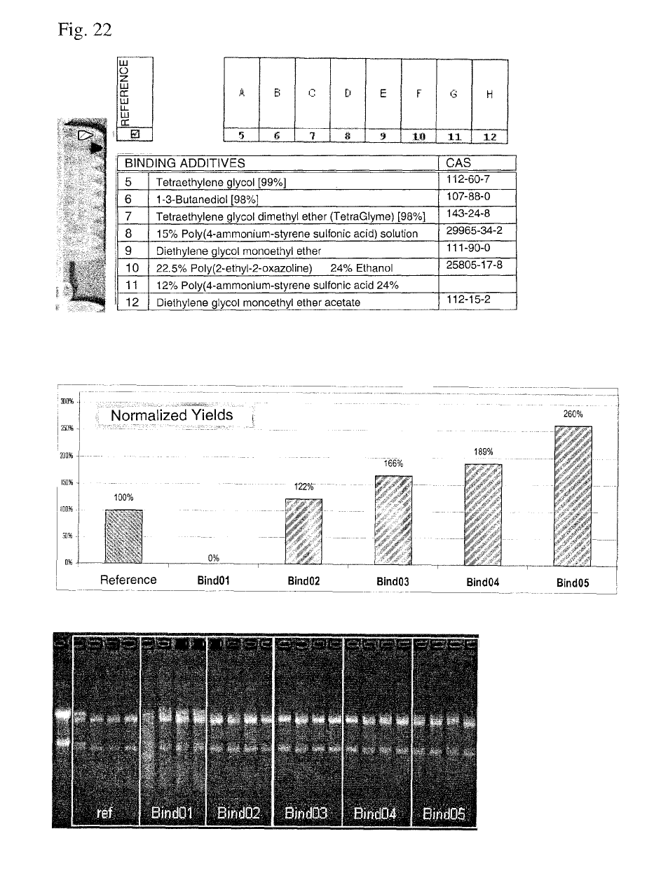

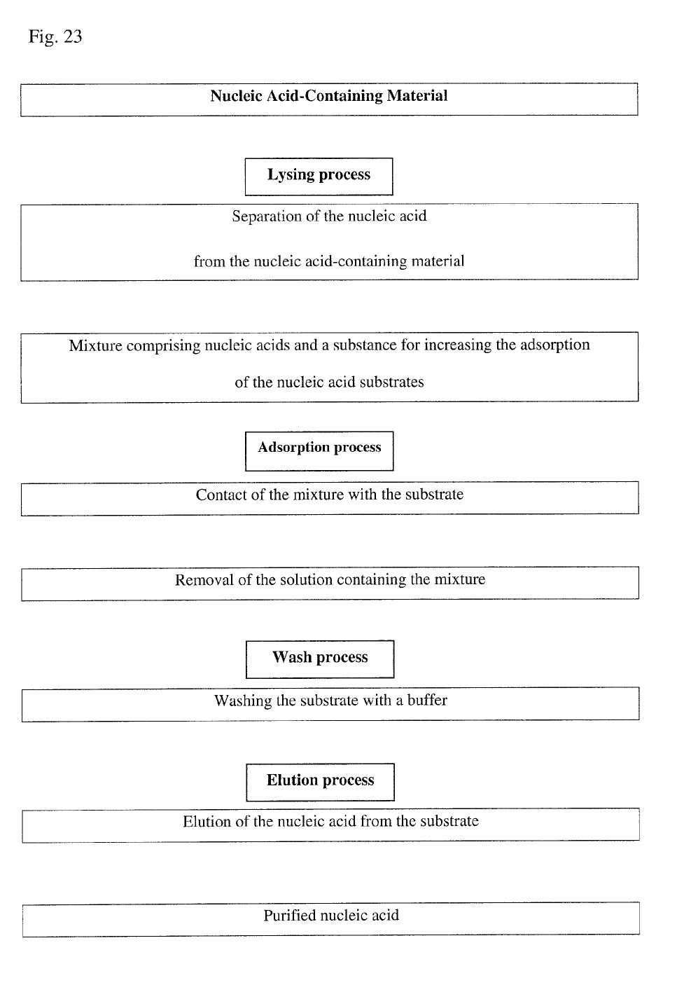

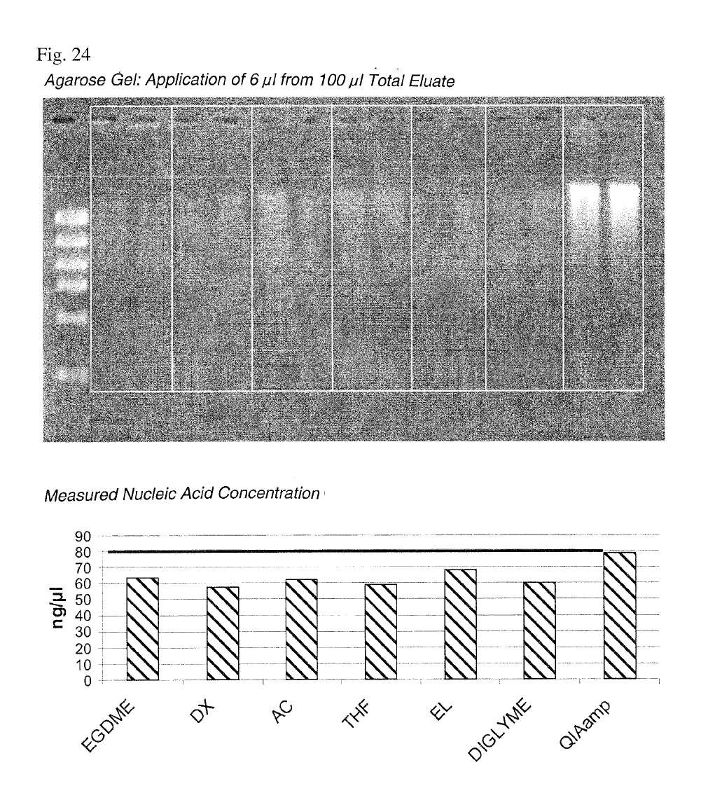

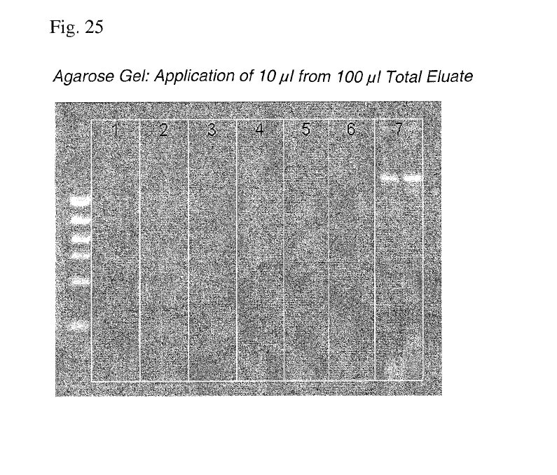

Upper table: normalized results determined by means of β-actin qPCR; lower table: agarose gel with the individual samples
1A: 13.5% poly(2-ethyl-2-oxazoline)
1B: 22.5% poly(2-ethyl-2-oxazoline); 24% ethanol

### 2A: 98.0% TetraGlyme

### 2B: 73.5% TetraGlyme

FIG. 2: Behavior of diethylene glycol monoethyl ether in the QIAamp® 96 Spin Blood Protocol.

Upper table: normalized results determined by means of β-actin qPCR; lower table: agarose gel with the individual samples
A: 99.0% diethylene glycol monoethyl ether
B: 74.3% diethylene glycol monoethyl ether; 24% ethanol
C, 61.9% diethylene glycol monoethyl ether; 24% ethanol
D: 80.0% diethylene glycol monoethyl ether; 16% ethanol

FIG. 3: Behavior of diethylene glycol monoethyl ether acetate in the QIAamp® 96 Spin Blood Protocol.

Table: normalized results determined by means of β-actin qPCR
A: 99.0% diethylene glycol monoethyl ether acetate
B: 74.3% diethylene glycol monoethyl ether acetate; 24% ethanol
C, 61.9% diethylene glycol monoethyl ether acetate; 24% ethanol
D: 80.0% diethylene glycol monoethyl ether acetate; 16% ethanol

FIG. 4: Behavior of the poly(4-ammonium-styrene sulfonic acid) solution in the QIAamp® 96 Spin Blood Protocol.

Upper table: normalized results determined by means of β-actin qPCR; lower table: agarose gel with the individual samples
A: 12% poly(4-ammonium-styrene sulfonic acid) solution
B: 10% poly(4-ammonium-styrene sulfonic acid) solution
C: 12% poly(4-ammonium-styrene sulfonic acid) solution
D: 10% poly(4-ammonium-styrene sulfonic acid) solution

FIG. 5: Behavior of poly(2-ethyl-2-oxazoline) and TetraGlyme in the BioSprint® 96 DNA Blood Protocol.

Upper table: normalized results determined by means of β-actin qPCR; lower table: agarose gel with the individual samples
1A: 13.5% poly(2-ethyl-2-oxazoline)
1B: 22.5% poly(2-ethyl-2-oxazoline); 24% ethanol

### 2A: 98.0% TetraGlyme

### 2B: 73.5% TetraGlyme

FIG. 6: Behavior of diethylene glycol monoethyl ether in the BioSprint® 96 DNA Blood Protocol.

Upper table: normalized results determined by means of β-actin qPCR; lower table: agarose gel with the individual samples
A: 99.0% diethylene glycol monoethyl ether
B: 74.3% diethylene glycol monoethyl ether; 24% ethanol
C, 61.9% diethylene glycol monoethyl ether; 24% ethanol
D: 80.0% diethylene glycol monoethyl ether; 16% ethanol

FIG. 7: Behavior of diethylene glycol monoethyl ether acetate in the BioSprint® 96 DNA Blood Protocol.

Upper table: normalized results obtained by means of β-actin qPCR; lower table: agarose gel with the individual samples
A: 99.0% diethylene glycol monoethyl ether acetate
B: 74.3% diethylene glycol monoethyl ether acetate; 24% ethanol
C: 61.9% diethylene glycol monoethyl ether acetate; 24% ethanol
D: 80.0% diethylene glycol monoethyl ether acetate; 16% ethanol

FIG. 8: Behavior of diethylene glycol monoethyl ether acetate in the BioSprint® 96 DNA Blood Protocol.

Upper table: normalized results obtained by means of β-actin qPCR; lower table: agarose gel with the individual samples
A: 12% poly(4-ammonium-styrene sulfonic acid) solution
B: 8% poly(4-ammonium-styrene sulfonic acid) solution
C: 12% poly(4-ammonium-styrene sulfonic acid) solution
D: 8% poly(4-ammonium-styrene sulfonic acid) solution

FIG. 9: QIAquick® Protocol and the resulting purification of the gel pilot 1 kb ladder. The first lane represents the unpurified marker, lane “a” comprises a fragment purified by QIAquick, which is used here as reference. Under the mentioned conditions, no significant losses have been observed with regard to the results and/or the size-dependent purification.
M) untreated, “Gel Pilot® 1 kb Ladder”
a) buffer PM;
b) 5M GuHCl, 100 mM Na—Ac, 12% poly(4-ammonium-styrene sulfonic acid)
c) 5M GuHCl, 100 mM Na—Ac, 12% poly(4-ammonium-styrene sulfonic acid); 20% isopropanol
d) 5M GuHCl, 100 mM Na—Ac, 13.5% poly(2-ethyl-2-oxazoline)
e) 5M GuHCl, 100 mM Na—Ac, 10% poly(2-ethyl-2-oxazoline); 20% ethanol
f) 5M GuHCl, 100 mM Na—Ac, 30% TetraGlyme
g) 5M GuHCl, 10 mM Tris-Cl pH 7.5, 30% TetraGlyme

FIG. 10: QIAquick purification of a mixture of plasmid DNA and oligonucleotides.
The oligonucleotides are removed by the alternative purification protocols.
   AM) Starting material: mixture of plasmid DNA and a DNA oligonucleotide
   a) buffer PM
   b) 5M GuHCl, 100 mM Na—Ac, 12% poly(4-ammonium-styrene sulfonic acid)

### c) 5M GuHCl, 100 mM Na—Ac, 30% TetraGlyme

### d) 5M GuHCl, 10 mM Tris-Cl pH 7.5, 30% TetraGlyme

FIG. 11: Behavior of poly(2-ethyl-2-oxazoline) and TetraGlyme in the BioSprint® 96 Tissue Protocol.

Upper table: normalized results obtained by means of mouse GAPDH qPCR; lower table: agarose gel
1A: 13.5% poly(2-ethyl-2-oxazoline)
1B: 22.5% poly(2-ethyl-2-oxazoline); 24% ethanol

### 2A: 98.0% TetraGlyme

2B: 73.5% TetraGlyme; 24% ethanol

FIG. 12: Behavior of diethylene glycol monoethyl ether acetate in the BioSprint® 96 Tissue Protocol.

Upper table: normalized results obtained by means of mouse GAPDH qPCR; lower table: agarose gel
A: 99.0% diethylene glycol monoethyl ether acetate
B: 74.3% diethylene glycol monoethyl ether acetate; 24% ethanol
C, 61.9% diethylene glycol monoethyl ether acetate; 24% ethanol
D: 80.0% diethylene glycol monoethyl etheracetat; 16% ethanol

FIG. 13: Behavior of diethylene glycol monoethyl ether in the BioSprint® 96 Tissue Protocol.

Upper table: normalized yields obtained by means of mouse GAPDH qPCR; lower table: agarose gel
A: 99.0% diethylene glycol monoethyl ether
B: 74.3% diethylene glycol monoethyl ether; 24% ethanol
C, 61.9% diethylene glycol monoethyl ether; 24% ethanol
D: 80.0% diethylene glycol monoethyl ether; 16% ethanol

FIG. 14: Behavior of TetraGlyme in the DNeasy® 96 Tissue Protocol

Table: normalized results obtained by means of mouse GAPDH qPCR

### A: 98.0% TetraGlyme

### B: 73.5% TetraGlyme

FIG. 15: Behavior of diethylene glycol monoethyl ether acetate in the DNeasy® 96 Tissue Protocol.

Table: normalized results obtained by means of mouse GAPDH qPCR
A: 99.0% diethylene glycol monoethyl ether acetate
B: 74.3% diethylene glycol monoethyl ether acetate; 24% ethanol
C, 61.9% diethylene glycol monoethyl ether acetate; 24% ethanol
D: 80.0% diethylene glycol monoethyl ether acetate; 16% ethanol

FIG. 16: Behavior of diethylene glycol monoethyl ether acetate in the DNeasy® 96 Tissue Protocol.

Upper table: normalized results obtained by means of mouse GAPDH qPCR; lower table: agarose gel
A: 99.0% diethylene glycol monoethyl ether
B: 74.3% diethylene glycol monoethyl ether; 24% ethanol
C, 61.9% diethylene glycol monoethyl ether; 24% ethanol
D: 80.0% diethylene glycol monoethyl ether acetate; 16% ethanol

FIG. 17: Behavior of diethylene glycol monoethyl ether acetate in the DNeasy® 96 Tissue Protocol.

Table: normalized results obtained by means of lamin RT-qPCR; the cells used were “293” and MCF7

FIG. 18: Behavior of different replacement chemicals for ethanol in the DNeasy® 96 Protocol.

Upper table: normalized results obtained by means of mouse GAPDH qPCR; lower table: agarose gel
Binding additive 01=12% poly(4-ammonium-styrene sulfonic acid) solution (failed in PCR)
Binding additive 02=98% TetraGlyme
Binding additive 03=73.5% TetraGlyme; 24% ethanol
Binding additive 04=99% diethylene glycol monoethyl ether acetate
Binding additive 05=80% diethylene glycol monoethyl ether acetate; 16% ethanol

FIG. 19: Experiment with regard to fragment size inhibition

RNeasy® inhibits small RNAs (5,8 S; tRNA; miRNA; . . . ) during purification. The exclusion size is about 150 base quantities. In this experiment it is demonstrated that the size inhibition of the test chemicals is comparable to the reference values of ethanol.
Binding additive 1=98% TetraGlyme;
Binding additive 2=80% diethylene glycol monoethyl ether acetate; 16% ethanol

FIG. 20: Cartridge alignment of the EZ1® DNA Blood 200 μl Reagent Cartridge

Buffer ML in position 1
m1D: 4.5 M GTC; 50 mM NH₄Cl; 45 mM Tris pH 7.5; 20 mM EDTA; 2.0% Triton-X-100
ml9: 4.5 M GTC; 1.0 M NaCl; 50 mM NH₄Cl; 45 mM Tris pH 7.5; 20 mM EDTA; 2.0% Triton-X-100
   MW1 replacement buffer in position 4
“2”: 49% 1,3-butanediol; 2.5 MGuHCl
   MW2 replacement buffer in positions 5+6
“B”: 60% 1,3-butanediol; 100 mM NaCl; 10 mM Tris-Cl pH 7.5

FIG. 21: Behavior of different ethanol replacement chemicals in the EZ1® DNA Blood 20 μl Protocol.

Upper left table: normalized results obtained by means of β-actin qPCR; right table: “Delta-Delta-CT” analysis of different sample starting amounts. In the calculation process, the measured Delta-CT is compared with the theoretical Delta-CT whereby the numerical value of the PCR inhibition degree is disclosed; lower table: agarose gel

FIG. 22: Behavior of different ethanol replacement chemicals in the first binding step of the EZ1®-RNA-Protocol.

Upper table: cartridge alignment of the EZ1® DNA Blood 200 μl reagent cartridge; middle table: normalized results obtained by means of MapK2 RT qPCR; lower table: agarose gel
Bind01=12% poly(4-ammonium-styrene sulfonic acid) solution (failed in RT qPCR)
Bind02=98% TetraGlyme
Bind03=73.5% TetraGlyme; 24% ethanol
Bind04=99% diethylene glycol monoethyl ether acetate
Bind05=80% diethylene glycol monoethyl ether acetate; 16% ethanol

FIG. 23: shows a possible embodiment of the method according to the invention

FIG. 24: Comparison of the binding additives used US 2004/167324 A1 with the prior art methods exemplified by the QIAamp® Blood Protocol.

1: EGDME (ethylene glycol dimethyl ether); 2: DX (1,4-dioxane); 3: AC (acetone); 4: THF (tetrahydrofuran); 5: EL (ethyl lactate); 6: DIGLYME (diethylene glycol dimethyl ether); 7: reference—MagAttract® Blood Protocol

FIG. 25: Comparison of the binding additives used in US 2004/167324 A1 with the methods of the prior art exemplified by the MagAttract® Blood Protocol.

1: EGDME (ethylene glycol dimethyl ether); 2: DX (1,4-dioxane); 3: AC (acetone); 4: THF (tetrahydrofuran); 5: EL (ethyl lactate); 6: DIGLYME (diethylene glycol dimethyl ether); 7: reference—MagAttract® Blood Protocol

TABLE 1
Commercially available products of the Company QIAGEN as used in the
Examples

| | |
|---|---|
| MagAttract ® | Suspension with magnetic particles |
| Suspension G | |
| Buffer PE | Wash buffer with weak organic base |
| Buffer AE | Low salt buffer |
| Buffer EB | Aqueous elution buffer |
| Buffer TE | Elution buffer; 10 mM TrisCl, 1 mM EDTH pH 8 |
| RNase-free Water | Ultrapure water, RNase-free |
| Buffer AL | Lysis buffer comprising guanidinium hydrochloride |
| Buffer RLT | Buffer comprising thiocyanate |
| Buffer ATL | Buffer comprising EDTA and SDS |
| Buffer ML | Buffer comprising guanidinium thiocyanate and t- |
| | octylphenoxy-polyoxy ethanol |
| Buffer AP1 | Buffer comprising EDTA and SDS |
| Buffer AW1 | Wash buffer comprising guanidinium hydrochloride |
| Buffer AW2 | Wash buffer comprising sodium azide |
| Buffer RW1 | Alcohol-containing buffer with guanidinium salt |
| Buffer RPE | Aqueous buffer |
| ProtK | Proteinase K |
| Buffer PM | Binding buffer comprising guanidinium chloride |
| | and 2-propanol |
| Buffer MW1 | Use buffer comprising guanidinium |
| Ethanol | hydrochloride and ethanol |
| Buffer MW2 | Buffer with lithium chloride and ethanol |
| Ethanol | |
| GTC | Guanidinium thiocyanate |
| MW1 Replacement | Use buffer comprising guanidinium hydrochloride |
| Buffer | |
| MW2 Replacement | Buffer with lithium chloride |
| Buffer | |
| RDD | RNAse-free buffer |
| AlAamp Spin | QiaAmp ® Spin Columns |
| K-AC | Potassium acetate |
| EGME | Ethylene glycol monomethyl ether |
| MagSep | Magnetic separation |
| MagStep | Step for magnetic separation |

The reagents and buffers listed in Table 1 as well as the protocols described therein are publications and commercially available products of the company QIAGEN GmbH, Hilden.

### EXAMPLE 1

### BioSprint® 96 DNA Blood

BioSprint® 96 with Protocol File: “BS96_DNA_Blut_{—}200”

### Lysis

200 μl blood
200 μl buffer AL
20 μl QIAGEN protease
incubation in a thermomixer for 15 min at 56° C. and 1400 rpm

### Binding

addition of 200 μl isopropanol to the standard reference protocol
isopropanol substitutes (add 200 μl each):
   1A) 98.0% TetraGlyme
   1B) 73.5% TetraGlyme; 24% ethanol
   2A) 99% diethylene glycol monoethyl ether acetate
   2B) 74.3% diethylene glycol monoethyl ether acetate; 24% ethanol
   2C) 61.9% diethylene glycol monoethyl ether acetate; 24% ethanol
   2D) 80% diethylene glycol monoethyl ether acetate; 16% ethanol
   3A) 12% poly(4-ammonium-styrene sulfonic acid) solution
addition of 30 μl MagAttract® Suspension G
Wash steps
   1× buffer AW1 (650 μl)
   1× buffer AW1 (500 μl)
   2× buffer AW2 (500 μl)
Rinsing with aqueous solution: 0.02% Tween® 20
Elution: 200 μl buffer TE in 96-well MicroTubePack MicroPlate

### EXAMPLE 2

### QIAamp® 96 Spin Blood Protocol

### Lysis

200 μl blood
200 μl buffer AL
20 μl QIAGEN protease
incubation 15 min at 56° C.

### Binding

Addition of 200 μl ethanol to the standard reference protocol
Ethanol substitutes (add 200 μl each):
   1A) 99% diethylene glycol monoethyl ether
   1B) 74.3% diethylene glycol monoethyl ether; 24% ethanol
   1 C) 61.9% diethylene glycol monoethyl ether; 24% ethanol
   1D) 80% diethylene glycol monoethyl ether; 16% ethanol
   2A) 99% diethylene glycol monoethyl ether acetate
   2B) 74.3% diethylene glycol monoethyl ether acetate; 24% ethanol
   2C) 61.9% diethylene glycol monoethyl ether acetate; 24% ethanol
   2D) 80% diethylene glycol monoethyl ether acetate; 16% ethanol
   3A) 98.0% TetraGlyme
   3B) 73.5% TetraGlyme; 24% ethanol
   4) 10% poly(4-ammonium-styrene sulfonic acid) solution; 24% ethanol
mix in 96-well deep-well block and transfer to QIAamp® 96 plate
   Wash steps
1× buffer AW1 (650 μl)
1× buffer AW2 (500 μl)
   Elution: 200 μl buffer TE in elution microtube rack

### EXAMPLE 3

### BioSprint® 96 DNA Tissue

### BioSprint® 96 Protocol File: “BS96_DNA_Blut_{—}200”

### Lysis

200 μl lysate (25 mg tissue+180 μl buffer ATL+20 μl proteinase K, overnight incubation at 56° C.)
addition of 200 μl buffer AL

### Binding

addition of 200 μl isopropanol to standard reference protocol
isopropanol substitutes (add 200 μl each):
   1A) 98.0% TetraGlyme
   1B) 73.5% TetraGlyme; 24% ethanol
   2A) 99% diethylene glycol monoethyl ether acetate
   2B) 74.3% diethylene glycol monoethyl ether acetate; 24% ethanol
   2C) 61.9% diethylene glycol monoethyl ether acetate; 24% ethanol
   2D) 80% diethylene glycol monoethyl ether acetate; 16% ethanol
   3A) 99% diethylene glycol monoethyl ether
   3B) 74.3% diethylene glycol monoethyl ether; 24% ethanol
   3C) 61.9% diethylene glycol monoethyl ether; 24% ethanol
   3D) 80% diethylene glycol monoethyl ether; 16% ethanol
+30 μl MagAttract Suspension G
   Wash steps
1× buffer AW1 (650 μl)
1× buffer AW1 (500 μl)
2× buffer AW2 (500 μl)
rinsing with aqueous solution: 0.02% Tween 20 (500 μl)
   Elution: 200 μl buffer TE in microtube plate

### EXAMPLE 4

### DNeasy® 96 Tissue

### Lysis

200 μl lysate (25 mg tissue+180 μl buffer ATL+20 μl proteinase K, overnight incubation at 56° C.)
addition of 200 μl buffer AL

### Binding

addition of 200 μl ethanol to the standard reference protocol
ethanol substitutes (add 200 μl each):
   1A) 99% diethylene glycol monoethyl ether
   1B) 74.3% diethylene glycol monoethyl ether; 24% ethanol
   1 C) 61.9% diethylene glycol monoethyl ether; 24% ethanol
   1D) 80% diethylene glycol monoethyl ether acetate; 16% ethanol
   2A) 99% diethylene glycol monoethyl ether acetate
   2B) 74.3% diethylene glycol monoethyl ether acetate; 24% ethanol
   2C) 61.9% diethylene glycol monoethyl ether acetate; 24% ethanol
   2D) 80% diethylene glycol monoethyl ether acetate; 16% ethanol
   3A) 98.0% TetraGlyme
   3B) 73.5% TetraGlyme; 24% ethanol
mix in 96-well deep well block and transfer to DNeasy® 96 plate
   Wash steps
1× buffer AW1 (650 μl)
1× buffer AW2 (500 μl)
   Elution: 200 μl buffer TE in elution microtube rack

### EXAMPLE 5

### RNeasy® 96

### Binding

350 μl buffer RLT-lysate (“293” cells; 2×10⁵ cells/sample)
addition of 350 μl ethanol to the standard reference protocol
ethanol substitutes (add 200 μl each):
   1) 80% diethylene glycol monoethyl ether acetate; 16% ethanol
   2) 98% TetraGlyme
mix in S block and transfer to the RNeasy® 96 plate
   Wash steps
2× buffer RW1 (650 μl)
2× buffer RPE (500 μl)
   Elution: 100 μl RNase-free water in elution microtube rack

### EXAMPLE 6

### QIAquick®

### Binding

1 volume nucleic acid-containing sample
+5 volumes buffer PM (standard reference protocol)
Substitute for buffer PM
   12% poly(4-ammonium-styrene sulfonic acid); 5M GuHCl; 100 mM sodium acetate
   30% TetraGlyme; 5M GuHCl; 10 mM Tris pH 7.5
   10% poly(2-ethyl-2-oxazoline), 5M GuHCl, 100 mM sodium acetate; 20% ethanol
loading the QIAamp® MinElute Spin Column; centrifugation for 1 min at 8,000 rpm
   Wash steps
1 wash step with buffer PE
“Dry Spin”
   Elution: 40 μl RNase-free water in elution microtube

### EXAMPLE 7

### EZ1® DNA Blood 200 μl Protocol

### EZ1® DNA Blood 200 μl Reagent Cartridge—List of Contents

| Filling | | | Replacement |
|---|---|---|---|
| Position | Contents | Amounts (μl) | Position |
| 1 | Lysis buffer (buffer ML) | 740 | [symbol] |
| 2 | “MagAttract Suspension B” | 300 | |
| 3 | “Bead buffer” | 60 | |
| 4 | Wash buffer I (buffer MW1 | 900 | [symbol] |
| | ethanol) | | |
| 5 | Wash buffer II (buffer MW2 | 900 | [symbol] |
| | ethanol) | | |
| 6 | Wash buffer II (buffer MW2 | 900 | [symbol] |
| | ethanol) | | |
| 7 | Rinse (ultrapure water) | 1000 | |
| 8 | Elution buffer (ultrapure water) | 220 | |
| 9 | empty | 0 | |
| 10 | empty | 1000 | |

Replacement buffers

| | Position |
|---|---|
| ML replacement buffer | |
| 4.5M GTC; 1.0M NaCl; 50 mM NH₄Cl; 45 mM Tris pH 7.5; | 1 |
| 20 mM EDTA; 2.0% Triton-X-100 | |
| 4.5M GTC; 50 mM NH₄Cl; 45 mM Tris pH 7.5; 20 mM | 1 |
| EDTA; 2.0% Triton-X-100 | |
| MW1 replacement buffer | |
| 49% 1,3-butanediol; 2.5M GuHCl | 4 |
| MW2 replacement buffer | |
| 60% 1,3-butanediol; 100 mM NaCl; 10 mM Tris-Cl pH 7.5 | 4 + 6 |

### EXAMPLE 8

### EZ1®-RNA Protocol

### EZ1® RNA Reagent Cartridge—List of Contents

| Filling | | Amount(s) | Replacement |
|---|---|---|---|
| position: | Content | (μl) | position |
| 1 | buffer RPE + 96% EtOH | 400 + 100 | [symbol] |
| | (=buffer RPE | | |
| | working solution) | | |
| 2 | 0.5M LiCl + “MagAttract | 320 + 80 | |
| | Suspension B” | | |
| 3 | buffer MW1 + 96% EtOH | 344 + 456 | [symbol] |
| | (=buffer AW1 | | |
| | working solution) | | |
| 4 | buffer RPE + 96% EtOH | 160 + 640 | [symbol] |
| | (=buffer RPE working | | |
| | solution) | | |
| 5 | buffer RDD | 245 | |
| 6 | buffer MW1 (=buffer AW1 | 250 | |
| | concentrate) | | |
| 7 | buffer MW1 + 96% EtOH | 251 + 785 | [symbol] |
| | (=buffer AW1 | | |
| | working solution 2) | | |
| 8 | buffer RPE + 96% EtOH | 180 + 270 | [symbol] |
| 9 | ultrapure water | 1000 | |
| 10 | ultrapure water | 200 | |

Replacement buffers

| | Cartridge |
|---|---|
| | position |
| Binding additives | |
| tetraethylene glycol (99%) | 1 |
| 1,3-butanediol (98%) | 1 |
| 80% diethylene glycol monoethyl ether acetate; 16% ethanol | 1 |
| Wash buffers | |
| 56% 1,3-butanediol; 3M GuHCl | 3 |
| 60% 1,3 butanediol; 100 mM NaCl; 10 mM Tris-Cl pH 7.5 | 4 |
| 65% tetraethylene glycol; 900 mM GTC; 10 mM Tris/Cl | 7 |
| pH 7.5 | |
| 60% 1,3 butanediol; 30 mM NaCl; 10 mM Tris-Cl pH 7.5 | 8 |

### EXAMPLE 9

### Comparison of the Binding Additives According to the Present Invention with Those of the Prior Art

Subject-Matter: Comparison of the organic solvents used in US 2004/167324 A1 (Hitachi) as binding additives for classical chaotropic bindings on silica with the reference binding additives for QIAamp® and MagAttract® used in accordance with the present invention

### Material:

Blood and buffers
Lysis buffer: 3 M GuHCl; 5% Triton X-100
Wash buffer: 25 mM potassium acetate; 50% ethanol
Elution buffer: buffer TE
Substitution reagents

| | | |
|---|---|---|
| 1 | EGME | ethylene glycol dimethyl ether |
| 2 | DX | 1,4-dioxane |
| 3 | AC | acetone |
| 4 | THF | tetrahydrofuran |
| 5 | EL | ethyl lactate |
| 6 | DIGLYME | diethylene glycol dimethyl ether |

Method: Preparation of Genomic DNA from 100 μl Blood Using QIAamp® Spin Columns:
1. 100 μl blood+10 μl proteinase K+100 μl lysis buffer
2. mixing and incubation for 10 min at 56° C.
3. addition of 100 μl substitution reagent and mixing
4. loading the lysate on the QIAamp® Spin Column; centrifugation for 30 sec at 8,000 rpm
5. washing with 3×500 μl wash buffer; each centrifugation for 30 sec at 8,000 rpm
6. “Dry-spin” for 1 min at 14,000 rpm
7. addition of 100 μl elution buffer, wait for 2 min and elute in new collection tube by centrifugation for at most 1 min
Control: QIAamp® Blood Mini carried out with 100 μl blood and eluted with 100 μl TE

### Results

### UV-Quantitation

| | Additive | OD260 | Mean | Conc ng/μl | Membrane Staining |
|---|---|---|---|---|---|
| 1 | EGDME | 0.266 | 0.254 | 63.38 | ++ |
| | | 0.241 | | | |
| 2 | DX | 0.229 | 0.231 | 57.63 | ++ |
| | | 0.232 | | | |
| 3 | AC | 0.244 | 0.248 | 62.00 | ++ |
| | | 0.252 | | | |
| 4 | THF | 0.24 | 0.235 | 58.75 | − |
| | | 0.23 | | | |
| 5 | EL | 0.266 | 0.274 | 68.50 | + |
| | | 0.282 | | | |
| 6 | DIGLYME | 0.24 | 0.241 | 60.25 | + |
| | | 0.242 | | | |
| 7 | QIAamp ® | 0.31 | 0.316 | 78.88 | − |
| | | 0.321 | | | |

Legend:
Membrane staining:
++: strongly stained;
+: slightly stained;
−: no staining

The results of the comparison are shown in FIG. 24.

In the given system, the organic solvents used in US 2004/167324 A1 failed as additives of DNA on silica membranes. On the agarose gel very low yields can be observed, while the UV OD measurements indicate an overquantitation.

Method: Preparation of Genomic DNA from 100 μl Blood Using Magnetic Silica Particles:

### MagBead® Procedure

1. 200 μl blood+20 μl proteinase K+200 μl lysis buffer
2. mix and incubate for 10 min at 56° C.
3. add 215 μl substitution reagent and 30 μl MagAttract Suspension A
4. shake in thermomixer; 5 min at 800 rpm; initially short mixing in the vortex mixer
5. magnetic separation in a suitable apparatus and removal of the supernatant
6. washing with 3×5,000 μl wash buffer
7. air drying of the magnetic particles
8. elution: 100 μl buffer TE; mixing for 1 min and magnetic separation. Supernatant contains the prepared genomic DNA and is transferred into a suitable vessel.

### Reference Method: MagAttract® Blood

1. 200 μl blood+20 μl QIAGEN protease+200 μl buffer AL
2. mixing and incubation (10 min at 56° C.)
3. addition of 200 μl isopropanol and 30 μl MagAttract® Suspension A
4. shake in thermomixer; 5 min at 800 rpm; initially short mixing in the vortex mixer
5. magnetic separation in a suitable apparatus and removal of the supernatant
6. washing with 500 μl buffer AW1 and 500 μl buffer AW2
7. air drying of the magnetic particles
8. elution: 100 μl buffer TE; mixing for 1 min and magnetic separation. Supernatant contains the prepared genomic DNA and is transferred into a suitable vessel.

As shown by the agarose gel in FIG. 25, the yields of genomic DNA are rather low for the samples which were prepared using magnetic silica particles and as binding additives the original solvents used in US 2004/167324 A1. The observed low yields according to “US 2004/167324 A1” are independent of the constitution of the adsorptive medium (magnetic silica particles or silica membranes).

1. Method for extracting nucleic acids from a solution, comprising the steps:
(a) adding a binding mediator to the nucleic acid-containing solution,
(b) contacting the solution comprising the binding mediator and the nucleic acids with a surface under chaotropic and/or high salt conditions,
(c) binding or adsorption of the nucleic acids to a surface,
(d) washing the surface with a washing buffer,
(e) recovering the nucleic acids which are adsorbed to the surface by elution, characterized in that the binding mediator is selected from the group comprising diethylene glycol monoethyl ether, diethylene glycol monoethyl ether acetate, furfuryl alcohol, poly(1-vinylpyrrolidone-co-2-dimethylaminoethylmethacrylate), poly(2-ethyl-2-oxazoline), poly(4-ammonium-styrene-sulfonic acid), tetraethylene glycol dimethyl ether, tetra ethylene glycol, tetrahydrofurfuryl-polyethylene glycol 200 and triethylene glycol monoethyl ether.

2. The method of claim 1 characterized in that the binding mediator is diethylene glycol monoethyl ether and is present in a concentration of 70 to 99 percent by volume.

3. The method of claim 1 or 2 characterized in that the surface to which the nucleic acids are adsorbed is based on materials that are selected from the following group: silica materials, carboxylated surfaces, zeolites and titanium dioxide.

4. The method of any one of the preceding claims characterized in that chaotropic conditions are achieved by the addition of chaotropic salts selected from the group comprising potassium iodide, guanidinium hydrochloride, guanidinium thiocyanate or sodium chloride to the nucleic acid-containing solution.

5. The method of any one of the preceding claims characterized in that the nucleic acid is genomic DNA.

6. The method of any one of the preceding claims characterized in that the nucleic acid is total RNA.

7. The method of any one of the preceding claims characterized in that the nucleic acids are short double-stranded DNA fragments.

8. The method of any one of the preceding claims characterized in that the nucleic acid-containing solution is obtained from a nucleic acid-containing material by a lysing process.

9. The method of any one of claims 1 to 8 characterized in that the nucleic acid-containing solution is obtained from a biochemical nucleic acid modification reaction.

10. The method of any one of claims 1 to 9 characterized in that the nucleic acid-containing material is selected from the group comprising blood, tissue, smear preparations, bacteria, cell suspensions and adherent cells, PCR reactions and in vitro-nucleic acid modification reactions.

11. Reagent kit for the extraction of nucleic acids from a solution, comprising
a solution 1 comprising the binding mediator selected from the group comprising diethylene glycol monoethyl ether, diethylene glycol monoethyl ether acetate, furfuryl alcohol, poly(1-vinylpyrrolidone-co-2-dimethylaminoethylmethacrylate), poly(2-ethyl-2-oxazoline), poly(4-ammonium-styrene sulfonic acid), tetraethylene glycol dimethyl ether, tetra ethylene glycol, tetrahydro-furfuryl-polyethylene glycol 200 and triethylene glycol monoethyl ether.

12. The reagent kit for the extraction of nucleic acids from a solution according to claim 11 characterized in that the binding mediator is diethylene glycol monoethyl ether and is present in a concentration of 70 to 99 percent by weight.

13. The reagent kit for the extraction of nucleic acids from a solution according to claim 11 or 12, further comprising
a solution 2 comprising wash buffer, and
a solution 3 comprising an eluant.

14. The reagent kit for the extraction of nucleic acids from a solution according to any one of claims 11 to 13 comprising a further solution 4 comprising a lysis buffer and a protease.

15. The reagent kit for the extraction of nucleic acids from a solution according to any one of claims 11 to 14 characterized in that at least one available lysing solution comprises a chaotropic salt.

16. The reagent kit for the extraction of nucleic acids from a solution according to claim 15 characterized in that the chaotropic salt is selected from a group comprising potassium iodide, guanidinium hydrochloride, guanidinium thiocyanate and sodium chloride.

17. Use of a reagent kit according to any one of claims 11 to 16 for the extraction of nucleic acids from biological materials selected from the group comprising blood, tissue, smear preparations, bacteria, cell suspensions and adherent cells.

18. Use of a reagent kit according to any of claims 11 to 16 for the purification of nucleic acids from biochemical reactions, PCR reactions or in vitro-nucleic acid modification reactions.
